SEQUENCE LISTING (14 sequences)

SEQ ID NO: 1 - DNA, 165 bp, Artificial sequence
  a nucleotide sequence encoding epidermal growth factor
atgaacagcgattcagaatgtccactgagccatgacggatactgcctgcacgacggcgtctgcatgtacatcgaggcactggacaagtacgcgtgcaactgtgttgttggatacatcggtgagcgttgtcaataccgtgatcttaagtggtgggaactgcgctga

SEQ ID NO: 2 - protein, 53 aa, Artificial sequence
  Synthetic Construct
MNSDSECPLSHDGYCLHDGVCMYIEALDKYACNCVVGYIGERCQYRDLKWWEL

SEQ ID NO: 3 - DNA, 30 bp, Artificial sequence
  Primer
cccaagctttcagcgcagttcccaccactt

SEQ ID NO: 4 - DNA, 30 bp, Artificial sequence
  Primer
cgggatccaacagcgattcagaatgtccac

SEQ ID NO: 5 - DNA, 32 bp, Artificial sequence
  Primer
cgggatccaccggtacgcgtagaatcgagacc

SEQ ID NO: 6 - DNA, 32 bp, Artificial sequence
  Primer
cggaattcatgaagtgggtaacctttatttcc

SEQ ID NO: 7 - DNA, 29 bp, Artificial sequence
  Primer
cgggatccgcgcagttcccaccacttaag

SEQ ID NO: 8 - DNA, 32 bp, Artificial sequence
  Primer
cggaattcatgaacagcgattcagaatgtcca

SEQ ID NO: 9 - DNA, 30 bp, Artificial sequence
  Primer
cccaagctttcaaccggtacgcgtagaatc

SEQ ID NO: 10 - DNA, 30 bp, Artificial sequence
  Primer
cgggatccaagtgggtaacctttatttccc

SEQ ID NO: 11 - DNA, 30 bp, Artificial sequence
  Primer
ctagctagcgatgaagtgggtaacctttat

SEQ ID NO: 12 - DNA, 30 bp, Artificial sequence
  Primer
ctagctagccgcagttcccaccacttaaga

SEQ ID NO: 13 - DNA, 30 bp, Artificial sequence
  Primer
ctagctagcgatgaacagcgattcagaatg

SEQ ID NO: 14 - DNA, 30 bp, Artificial sequence
  Primer
ctagctagcccggtacgcgtagaatcgaga

(19) United States
(12) Patent Application Publication
(10) Pub. No.: US 2006/0195945 A1
(43) Pub. Date: Aug. 31, 2006

(54) Method for preparing fusion polypeptide comprising epidermal growth factor and human serum albumin in plants

(76) Inventors: Sun Lee, Daejon Metropolitan City (KR); Jae-Geun Yoo, Daejon Metropolitan City (KR); Sun-Hee Park, Gyeonggi-do (KR)

Correspondence Address: JHK Law, P O Box 1078, La Canada, CA, 91012-1078, US

(21) Appl. No.: 10/520,388

(22) PCT Filed: Jul. 2, 2003
(86) PCT No.: PCT/KR03/01310

§ 371 Date: Nov. 7, 2005

(30) Foreign Application Priority Data
Jul. 3, 2002 (KR) 10-2002-38165

(51) Int. Cl. A01H 1/00; C12N 5/04; C12P 21/04; C12N 15/87; C07K 14/475

(52) U.S. Cl. 800/288; 800/294; 435/69.7; 435/419

(57) ABSTRACT

The present invention relates to a method for preparing a fusion polypeptide comprising epidermal growth factor (EGF) and human serum albumin in a plant.

### BACKGROUND OF THE INVENTION

1. Field of the Invention

The present invention relates to a method for preparing a fusion polypeptide comprising epidermal growth factor (EGF) and human serum albumin in a plant.

2. Description of the Related Art

Epidermal growth factor (hereinafter referred to as “EGF”) has been found to have several practical uses. For example, as applied to a portion observed to have ulcer symptom such as diabetic tinea ulcer and decubital ulcer, EGF promotes skin regeneration and then prevent aggravation of the condition. In addition, EGF has been reported to have a treatment effect to chronic skin ulcer and gastric ulcer. EGF has been used for minimization of scar associated with cornea damage and operation. Furthermore, EGF has been administered to patient suffered from burn for skin generation. In cosmetics for preventing senescence, EGF has been employed as active ingredient for reducing wrinkles and promoting skin regeneration. Hence, many researches have been made in order to obtain EGF.

First, it has been proposed that EGF could be purified from natural source such as human urine (Gregory, H. et al., Hoppe Seylers Z Physiol Chem. 356(11):1765-74(1975); and Savage C R Jr. et al., Anal Biochem. 111(1):195-202(1981)). However, this approach shows lower yield due to frequent precipitation and condensation steps in purification process and is not suitable in massive production.

Secondly, E. coli (Smith et al., J Gen Microbiol., 128(Pt 2):307-18(1982); and Taniyama et al., Jpn J Cancer Res. 77(2): 145-52(1986)), Bacillus subtilis (Yamagata, Proc Natl Acad Sci U S A. 86(10):3589-93(1989)) or yeast (Urdea et al., Proc Natl Acad Sci U S A. 80(24):7461-5(1983)) has been transformed with a gene encoding EGF and then subject to expression of EGF. However, this technology has some disadvantages. For example, EGF expressed in host cell is very likely to be degraded by endogenous protease, so that its expression level and yield is too low. In addition, according to this method, several steps such as high performance liquid chromatography are necessary for producing high-purity EGF, thereby highly increasing the price of EGF.

To protect EGF expressed in host from an action of protease, an expression vector for secreting EGF outside host cell has been designed (Korean Pat. No. 102993). E. coli harboring the expression vector permits to overcome problems ascribed to endotoxin and contaminations from other cellular proteins. In addition, this method comprises a complicated process for purifying EGF including reverse phase chromatography, anion exchange chromatography and reverse phase high performance liquid chromatography using C₁₈ column. However, this technology has been proved to cause high production cost due to the complexity of purification process and to give rise to unstable EGF.

Finally, a protein fusion technology has been employed for the production of EGF. However, after production of the fusion protein comprising EGF, it requires the use of endopeptidase acting at the fusion site for obtaining active and pure EGF.

Throughout this application, several patents and publications are referenced and citations are provided in parentheses. The disclosure of these patents and publications is incorporated into this application in order to more fully describe this invention and the state of the art to which this invention pertains.

### SUMMARY OF THE INVENTION

Endeavoring to resolve the problems of such conventional approaches, the inventor has made intensive research to develop a novel method for preparing EGF with higher stability in more convenient manner, particularly, by use of a plant bioreactor. As a result, the inventors have found that EGF coupled to albumin, particularly, human serum albumin showed considerable stability and prepared conveniently in pure form when using a plant bioreactor for producing EGF.

Accordingly, it is an object of this invention to provide a fusion polypeptide comprising epidermal growth factor and human serum albumin.

It is another object of this invention to provide a nucleotide sequence encoding the fusion polypeptide.

It is still another object of this invention to provide an expression vector comprising the nucleotide sequence encoding the fusion polypeptide.

It is further object of this invention to provide a transformant comprising the nucleotide sequence encoding the fusion polypeptide.

It is still further object of this invention to provide a method for preparing the fusion polypeptide.

It is another object of this invention to provide a method for preparing the fusion polypeptide in a plant.

It is still another object of this invention to provide a cosmetic composition for skin care.

It is further object of this invention to provide a pharmaceutical composition.

Other objects and advantages of the present invention will become apparent from the detailed description to follow taken in conjugation with the appended claims.

### BRIEF DESCRIPTION OF THE DRAWINGS

FIG. 1 schematically shows the cloning procedure of alumin-EGF in E. coli.

FIG. 2 schematically shows the cloning procedure of EGF-alumin in E. coli.

FIG. 3 shows the genetic map of pET28α carrying the nucleotide sequence encoding the fusion protein of this invention.

FIG. 4 shows the result of the electrophoresis of crude extracts from transformant on polyacrylamide gel.

FIG. 5 shows the result of the electrophoresis of purified fusion protein on polyacrylamide gel.

FIG. 6 shows the result of Western Blotting of purified fusion protein by use of anti-EGF antibody.

FIG. 7 is a genetic map of the binary vector used in this invention for a plant.

FIG. 8 represents the result of PCR amplification for verifying transformation of plants with albumin-EGF fusion gene.

FIG. 9 represents the result of PCR amplification for verifying transformation of plants with EGF-albumin fusion gene.

FIG. 10 shows the result of the electrophoresis of crude extract from plant transformants on polyacrylamide gel.

FIG. 11 shows the result of Western Blotting of the fusion protein from plant transformants by use of anti-EGF antibody.

### DETAILED DESCRIPTION OF THIS INVENTION

In one aspect of this invention, there is provided a fusion polypeptide comprising epidermal growth factor (EGF) and human serum albumin linked to the C-terminal or N-terminal of EGF; and in which the stability of EGF is enhanced by virtue of human serum albumin.

The present inventors have made intensive researches to develop a novel method for preparing EGF with higher stability in more convenient manner, particularly, by use of a plant bioreactor. As a result, the inventors have found that EGF coupled to albumin, particularly, human serum albumin showed considerable stability and prepared conveniently in pure form when using a plant bioreactor for producing EGF.

The term “epidermal growth factor” (abbreviated as EGF) used herein refers to human epidermal growth factor unless otherwise indicated. Human EGF itself is a 53 amino acid polypeptide and its analogs vary in the number of amino acids in the polypeptide chain. A variety of these have described in U.S. Pat. No. 3,917,824. Most preferably, human EGF is a polypeptide having an amino acid sequence as set forth in SEQ ID NO:2.

The term “albumin” used herein refers to human serum albumin (abbreviated as HSA) unless otherwise indicated.

According to a preferred embodiment, human serum albumin is linked to the C-terminal of EGF. As demonstrated in Example XII, EGF in the fusion protein, EGF-HSA is much more stable than EGF in HSA-EGF.

The expression “EGF-HSA” used herein refers to the fusion protein comprising EGF and HSA linked to the C-terminal of EGF. The expression “HSA-EGF” used herein refers to the fusion protein comprising EGF and HSA linked to the N-terminal of EGF. The symbol “-” between EGF and HSA is a linkage (covalent bond) formed between EGF and HSA. Such linkage also is expressed herein as attachment, coupling or fusion. EGF may be linked via an artificial peptide or preferably, directly to HSA.

The term “stability” with reference to EGF means that EGF maintains its inherent activity, i.e., mitogenic activity over time under certain conditions or environment.

EGF attached to its fusion partner HSA exhibits much higher stability than original EGF. In addition, EGF coupled to its fusion partner HSA manifests its activity without being interfered by its fusion partner HSA. HSA is non-immunogenic for human to which the fusion protein is applied.

In another aspect of this invention, there is provide a nucleotide sequence encoding a fusion polypeptide comprising EGF and human serum albumin linked to the C-terminal or N-terminal of EGF.

According to a preferred embodiment, a nucleotide sequence coding for human serum albumin is linked to the 3′-end of said EGF, so that EGF-HSA can be produced.

According to a preferred embodiment, a nucleotide sequence coding for EGF comprises nucleotide 1-159 as set forth in SEQ ID NO:1. This nucleotide sequence is newly prepared by the present inventors with modifying the known nucleotide sequence of EGF. More specifically, the present novel sequence is designed to (i) have codon usage suitable in expression in either a bacterium or a plant cell, particularly, a plant cell; (ii) have GC content of about 55%; and (iii) avoid intron or intron-like sequences in a plant. This novel nucleotide sequence of EGF is significantly advantageous in expression in a plant cell.

In still another aspect of this invention, there is provided an expression vector comprising the nucleotide sequence encoding the present fusion protein described above and a promoter operably linked to said nucleotide sequence. The term “operably linked” refers to functional linkage between a nucleic acid expression control sequence (such as a promoter, signal sequence, or array of transcription factor binding sites) and a second nucleotide sequence, wherein the expression control sequence affects transcription and/or translation of the nucleic acid corresponding to the second sequence.

According to a preferred embodiment of this invention, a nucleotide sequence of EGF comprises nucleotide 1-159 as set forth in SEQ ID NO:1.

The vector system of this invention may be constructed according to the known methods in the art as described in Sambrook et al., Molecular Cloning, A Laboratory Manual, Cold Spring Harbor Laboratory Press (2001), which is incorporated herein by reference.

Typically, the vector may be constructed for cloning or expression. In addition, the vector may be constructed for use in prokaryotic or eukaryotic host cells.

For example, where the vector is constructed for expression in prokaryotic cells, it generally carries a strong promoter to initiate transcription (e.g., pLλ promoter, trp promoter, lac promoter, tac promoter and T7 promoter), a ribosome binding site or translation initiation and a transcription/translation termination sequence. In particular, where E. coli is used as a host cell, a promoter and operator in operon for tryptophan biosynthesis in E. coli (Yanofsky, C., J. Bacteriol., 158:1018-1024(1984)) and a leftward promoter of phage λ (pLλ promoter, Herskowitz, I. and Hagen, D., Ann. Rev. Genet., 14:399-445(1980)) may be employed as a control sequence. Where Bacillus is used as a host cell, a promoter for a gene encoding toxin protein of Bacillus thurigensis (Appl. Environ. Microbiol. 64:3932-3938(1998); and Mol. Gen. Genet. 250:734-741(1996)) or other promoters operable in Bacillus may be employed as a control sequence.

Numerous conventional vectors used for prokaryotic cells are known to those of skill in the art, and the selection of an appropriate vector is a matter of choice. Conventional vector used in this invention includes pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, λgt4·λB, λ-charon, λΔz1 and M13, but not limited to.

For example, where the expression vector is constructed for eukaryotic host cell, inter alia, animal cell, a promoter derived the genome of mammalian cells (e.g., metallothionein promoter) or mammalian virus (e.g., adenovirus late promoter; vaccinia virus 7.5K promoter, SV40 promoter, cytomegalovirus promoter and tk promoter of HSV) may be used. The vector generally contains a polyadenylation site of the transcript. The example of commercial virus-based vectors includes pcDNA 3 (Invitrogen; containing cytomegalo virus promoter and polyadenylation signal), pSI (Promega; containing SV 40 promoter and polyadenylation signal), pCI (Promega; containing containing cytomegalo virus promoter and polyadenylation signal), and pREP7 (Invitrogen; RSV promoter and SV 40 polyadenylation signal).

Where the expression vector is constructed for yeast, the promoter of the gene for phosphoglycerate kinase, glyceraldehydes-3-phosphate dehydrogenase, lactase, enolase and alcohol dehydrogenase may be used as a control sequence.

Where the expression vector is constructed for a plant cell, numerous plant-functional promoters known in the art may be used, including the cauliflower mosaic virus (CaMV) 35S promoter, the Figwort mosaic virus 35S promoter, the sugarcane bacilliform virus promoter, the commelina yellow mottle virus promoter, the light-inducible promoter from the small subunit of the ribulose-1,5-bis-phosphate carboxylase (ssRUBISCO), the rice cytosolic triosephosphate isomerase (TPI) promoter, the adenine phosphoribosyltransferase (APRT) promoter of Arabidopsis, the rice actin 1 gene promoter, and the mannopine synthase and octopine synthase promoters.

In addition, the expression vector of this invention further comprises a nucleotide sequence to conveniently purify the fusion protein expressed, which includes but not limited to, glutathione S-transferase (Pharmacia, USA), maltose binding protein (NEB, USA), FLAG (IBI, USA) and 6X His (hexahistidine; Quiagen, USA). The most preferable sequence is 6X His because it has not antigenicity and does not interfere desirable folding of the fusion protein of interest. Due to the additional sequence, the fusion protein expressed can be purified with affinity chromatography in a rapid and feasible manner.

According to a preferred embodiment of this invention, the fusion protein is purified by affinity chromatography. For example, in case of using glutathione S-transferase, elution buffer containing glutathione is employed and in case of using 6X His, Ni-NTA His-binding resin (Novagen, USA) is generally employed to purify the fusion protein of interest in a rapid and feasible manner.

It is preferable that the expression vector of this invention carries one or more markers which make it possible to select the transformed host, for example, genes conferring the resistance to antibiotics such as ampicillin, gentamycine, chloramphenicol, streptomycin, kanamycin, neomycin, geneticin and tetracycline, URA3 gene, genes conferring the resistance to any other toxic compound such as certain metal ions.

In further aspect of this invention, there is provided a transformant harboring the nucleotide sequence encoding the present fusion protein described above.

The hosts useful in preparing the transformant are well known to those skilled in the art. For example, as prokaryotic host, E. coli JM109, E. coli BL21, E. coli RR1, E. coli LE392, E. coli B, E. coli X 1776, E. coli W3110, Bacillus subtilis, Bacillus thurigensis, Salmonella typhimurium, Serratia marcescens and various Pseudomonas, Corynebacterium and Streptomyces may be employed. As eukaryotic cell, yeast (Saccharomyce cerevisiae), insect cell, human cell (e.g., CHO, W138, BHK, COS-7, 293, HepG2, 3T3, RIN and MDCK cell lines) and plant cell may be used.

The transformation of a host cell can be carried out by a large number of methods known to one skilled in the art. For example, in case of using prokaryotic cells as host, CaCl₂ method (Cohen, S. N. et al., Proc. Natl. Acac. Sci. USA, 9:2110-2114(1973)), Hanahan method (Cohen, S. N. et al., Proc. Natl. Acac. Sci. USA, 9:2110-2114(1973); and Hanahan, D., J. Mol. Biol., 166:557-580(1983)) and electrophoresis (Dower, W. J. et al., Nucleic. Acids Res., 16:6127-6145(1988)) can be used for transformation. Also, in case of using eukaryotic cells as host, microinjection (Capecchi, M. R., Cell, 22:479(1980)), calcium phosphate precipitation (Graham, F. L. et al., Virology, 52:456(1973)), electroporation (Neumann, E. et al., EMBO J., 1:841(1982)), liposome-mediated transfection (Wong, T. K. et al., Gene, 10:87(1980)), DEAE-dextran treatment (Gopal, Mol. Cell Biol., 5:1188-1190(1985)), and particle bombardment (Yang et al., Proc. Natl. Acad. Sci., 87:9568-9572(1990)) can be use for transformation. In addition, where a plant cell is used as a host cell, Agrobacterium-mediated transformation is the most preferable because it is possible to bypass the need for regeneration of an intact plant from a protoplast (U.S. Pat. Nos. 5,004,863, 5,349,124 and 5,416,011).

In still further aspect of this invention, there is provided a method for preparing the fusion polypeptide comprising EGF and human serum albumin, which comprises the steps of: (a) culturing the transformant described above under conditions for expression; and (b) recovering the fusion polypeptide produced.

A variety of the methods for culturing the transformant are known to those skilled in the art as described in Sambrook et al., Molecular Cloning, A Laboratory Manual, Cold Spring Harbor Laboratory Press (2001), which is incorporated herein by reference. The recovery may be carried out, either during the cell growth for the continuous processes, or at the end of growth for the batch cultures.

According to a preferred embodiment, the recovery may be performed for obtaining the fusion protein in purified form. For example, where the fusion protein is expressed by the transformed bacteria in large amounts, typically after promoter induction, but expression can be constitutive, the protein may form insoluble aggregates (i.e., inclusion bodies). There are several protocols that are suitable for purification of inclusion bodies. The fusion proteins that form the inclusion bodies may then be renatured by dilution or dialysis with a compatible buffer. Thereafter, the fusion proteins may be purified in accordance with the standard methods known in the art including solubility fractionation by use of ammonium sulfate, size differential filtration (ultrafiltration) and column chromatography (based on size, net surface charge, hydrophobicity or affinity).

In another aspect of this invention, there is provided a method for preparing the fusion polypeptide comprising EGF and human serum albumin in a plant, which comprises the steps of: (a) transforming plant cells with the a polynucleotide sequence comprising: (i) the nucleotide sequence encoding the fusion protein described above; (ii) a promoter that functions in plant cells to cause the production of an RNA molecule operably linked to the nucleotide sequence of (i); and (iii) a 3′-non-translated region that functions in plant cells to cause the polyadenylation of the 3′-end of said RNA molecule; (b) selecting transformed plant cells; (c) regenerating a plant from the transformed cells; and (d) recovering the fusion polypeptide from the regenerated plant.

The nucleotide sequence encoding for the fusion protein described previously is much more suitable for expression in a plant compared to other hosts. In addition, the expression of the fusion protein in a plant has several advantages compared to the expression in bacterial hosts. Firstly, the fusion protein expressed in bacterial hosts generally forms insoluble inclusion body as described above, which requires complex and cost- and time-consuming procedures for obtaining purified fusion protein with its inherent activity. However, according to the present method, the fusion protein expressed in a plant is very likely to be soluble and active; therefore, the present method can provide cost- and time-effective approach for obtaining purified fusion protein with its inherent activity. Secondly, the transformed plant containing the fusion protein itself can be employed as a raw material. Thirdly, the plant producing the fusion protein exhibits no toxicity and is harmless to human. Finally, the plant as a bioreactor allows to simplify the production system and reduce the production cost significantly.

The 3′-non-translated region used in this invention may include that from the nopaline synthase gene of Agrobacterium tumefaciens (nos 3′ end) (Bevan et al., Nucleic Acids Research, 11(2):369-385(1983)), that from the octopine synthase gene of Agrobacterium tumefaciens, the 3′-end of the protease inhibitor I or II genes from potato or tomato, the CaMV 35S terminator.

The transformation of plant cells may be carried out according to the conventional methods known one of skill in the art, including electroporation (Neumann, E. et al., EMBO J., 1:841(1982)), particle bombardment (Yang et al., Proc. Natl. Acad. Sci., 87:9568-9572(1990)) and Agrobacterium-mediated transformation (U.S. Pat. Nos. 5,004,863, 5,349,124 and 5,416,011). Among them, Agrobacterium-mediated transformation is the most preferable. Agrobacterium-mediated transformation is generally performed with leaf disks and other tissues such as cotyledons and hypocotyls. This method is the most efficient in dicotyledonous plants.

The selection of transformed cells may be carried out with exposing the transformed cultures to a selective agent such as a metabolic inhibitor, an antibiotic and herbicide. Cells which have been transformed and have stably integrated a marker gene conferring resistance to the selective agent will grow and divide in culture. The exemplary marker includes, but not limited to, a glyphosphate resistance gene and a neomycin phosphotransferase (nptII) system.

The development or regeneration of plants from either plant protoplasts or various explants is well known in the art. The resulting transgenic rooted shoots are planted in an appropriate plant growth medium. The development or regeneration of plants containing the foreign gene of interest introduced by Agrobacterium may be achieved by methods well known in the art (U.S. Pat. Nos. 5,004,863, 5,349,124 and 5,416,011).

Meanwhile, the present inventors have made attempts to develop novel transformed plants such as Nicotiana tabacum, Cucumis melo, Curcumis saliva, Citrullus vulgaris and Brassica campestris and as a result, have established the most efficient methods for the transformation of certain plant. Such methods have been filed for patent application (PCT/KR02/01461, PCT/KR02/01462 and PCT/KR02/01463).

According to a preferred embodiment, the plant to be transformed is. Nicotiana tabacum, Cucumis melo, Curcumis sativa, Citrullus vulgaris and Brassica campestris.

According to a preferred embodiment, a nucleotide sequence of EGF comprises nucleotide 1-159 as set forth in SEQ ID NO:1.

In still another aspect of this invention, there is provided a method for preparing the fusion polypeptide comprising EGF and human serum albumin in a plant, which comprises the steps of: (a) inoculating an explant material from the plant with Agrobacterium tumefaciens harboring a vector, in which the vector is capable of inserting into a genome of a cell from the plant and contains the following nucleotide sequences: (i) the nucleotide sequence encoding the fusion protein described above; (ii) a promoter that functions in plant cells to cause the production of an RNA molecule operably linked to the nucleotide sequence of (i); and (iii) a 3′-non-translated region that functions in plant cells to cause the polyadenylation of the 3′-end of the RNA molecule; (b) regenerating the inoculated explant material on a regeneration medium to obtain regenerated shoots; (c) culturing the regenerated shoots on a rooting medium to obtain a transformed plant, in which the transformed plant is capable of expressing the nucleotide sequence encoding the fusion protein described above; and (d) recovering from the transformed plant the fusion polypeptide.

In this invention, the preferred explant for transformation includes any tissue derived from seed germinated. It is preferred to use cotyledon and hypocotyl and the most preferred is cotyledon. Seed germination may be performed under suitable dark/light conditions using an appropriate medium.

Transformation of plant cells derived is carried out with Agrobacterium tumefaciens harboring Ti plasmid (Depicker, A. et al., Plant cell transformation by Agrobacterium plasmids. In Genetic Engineering of Plants, Plenum Press, New York (1983)). More preferably, binary vector system such as pBin19, pRD400 and pRD320 is used for transformation (An, G. et al., Binary vectors” In Plant Gene Res. Manual, Martinus Nijhoff Publisher, New York (1986)). The binary vector useful in this invention carries: (i) a promoter capable of operating in plant cell; (ii) a structural gene operably linked to the promoter; and (iii) a polyadenylation signal sequence. The vector may alternatively further carry a gene coding for reporter molecule (for example, luciferase and β-glucuronidase) . Examples of the promoter used in the binary vector include but not limited to cauliflower mosaic Virus 35S promoter, 1′ promoter, 2′ promoter and promoter nopaline synthetase (nos) promoter.

Inoculation of the explant with Agrobacterium tumefaciens involves procedures known in the art. Most preferably, the inoculation involves immersing the cotyledon in the culture of Agrobacterium tumefaciens to coculture. Agrobacterium tumefaciens is infected into plant cells.

The explant transformed with Agrobacterium tumefaciens is regenerated in a regeneration medium, which allows successfully the regeneration of shoots. The transformed plant is finally produced on a rooting medium by rooting of regenerated shoots.

The transformed plant produced according to the present invention may be confirmed using procedures known in the art. For example, using DNA sample from tissues of the transformed plant, PCR is carried out to elucidate exogenous gene incorporated into a genome of the transformed plant. Alternatively, Northern or Southern Blotting may be performed for confirming the transformation as described in Maniatis et al., Molecular Cloning, A Laboratory Manual, Cold Spring Harbor Laboratory, Cold Spring Harbor, N.Y. (1989).

According to a preferred embodiment, the plant to be transformed is Nicotiana tabacum, Cucumis melo, Curcumis sativa, Citrullus vulgaris or Brassica campestris.

According to a preferred embodiment, the nucleotide sequence encoding EGF comprises nucleotides 1-159 as set forth in SEQ ID NO:1.

In further aspect of this invention, there is provided a cosmetic composition for skin care, which comprises: (a) a fusion polypeptide comprising EGF and human serum albumin linked to the C-terminal or N-terminal of EGF as an active ingredient; and (b) a cosmetically acceptable carrier.

In still further aspect of this invention, there is provided a pharmaceutical composition, which comprises: (a) a pharmaceutically effective amount of a fusion polypeptide comprising EGF and human serum albumin linked to the C-terminal or N-terminal of EGF as an active ingredient; and (b) a pharmaceutically acceptable carrier.

Human EGF has mitogenic activity for a number of kinds of cells, including epithelial and mesenchymal cells. EGF has been reported to be useful in increasing the rate of wound healing as a result of its mitogenic effect. EGF has also been reported as being useful for treating gastric ulcers. A review of EGF is provided by Carpenter et al., in Epidermal Growth Factor, Its Receptor and Related Proteins, Experimental Cell Research, 164:1-10(1986).

An important objective in the therapeutic use of EGF is the development of a stable cosmetic/pharmaceutical EGF formulation that has a long shelf life and is capable of remaining as a predominantly active species of EGF over a long period of time. However, because of the inherent instability of EGF, difficulties have been encountered in developing such a stable EGF formulation. For instance, EGF loses biological activity in the presence of moisture. Human EGF loses activity over time and produces multiple species of the EGF molecule, which have been identified by high performance liquid chromatography. These multiple species of EGF are believed to be breakdown products resulting from the degradation of EGF. Incubation of EGF at 45° C. accelerates the formation of the degradation products normally found with long term storage at ambient temperature. Such degradation, and the associated loss of biological activity of EGF, is a disadvantage because it makes it impractical to store aqueous or solid preparations of EGF over extended periods of time. In the cosmetic field, EGF has been widely employed for skin care.

The fusion protein of this invention itself can be used as active ingredient without cleavage treatment for obtaining EGF per se. In other words, EGF in the fusion protein can exhibit its mitogenic activity. In addition, the fusion protein shows suitable solubility, so that it is suitable in producing a cosmetic formulation. Importantly, EGF in the fusion protein shows much higher stability than non-fused EGF, so that the cosmetic composition comprising the fusion protein can maintain its efficacy for extended period of time.

According to a preferred embodiment, the fusion protein is prepared in a plant according to the present method described above. More preferably, the fusion protein in the cosmetic composition is prepared in Agrobacterium-mediated plant transformant according to the present method described above.

According to a preferred embodiment, in the fusion protein, human serum albumin is linked to the C-terminal of EGF.

The cosmetic compositions of this invention may be formulated in a wide variety of form, for example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these substances. In the formulation of powder or spray, it may comprise lactose, talc, silica, aluminum hydroxide, calcium silicate, polyamide powder and mixtures of these substances. Spray may additionally comprise the customary propellants, for example, chlorofluorohydrocarbons, propane/butane or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butylglycol, oils, in particular cottonseed oil, groundnut oil, maize germ oil, olive oil, castor oil and sesame seed oil, glycerol fatty esters, polyethylene glycol and fatty acid esters of sorbitan or mixtures of these substances. The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these substances.

The formulation of soap may comprise alkali metal salts of fatty acids, salts of fatty acid hemiesters, fatty acid protein hydrolyzates, isethionates, lanolin, fatty alcohol, vegetable oil, glycerol, sugars or mixtures of these substances.

Furthermore, the cosmetic compositions of this invention, may contain auxiliaries as well as carrier. The non-limiting examples of auxiliaries include preservatives, antioxidants, stabilizers, solubilizers, vitamins, colorants, odor improvers or mixtures of these substances

In the pharmaceutical compositions of this invention, the pharmaceutically acceptable carrier may be conventional one for formulation, including lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxy benzoate, propylhydroxy benzoate, talc, stearic acid, magnesium and mineral oil, but not limited to. The pharmaceutical compositions of this invention, further may contain wetting agent, sweetening agent, emulsifying agent, suspending agent, preservatives, flavors, perfumes, lubricating agent, or mixtures of these substances.

The pharmaceutical compositions of this invention, may be administered orally or parenterally. The topical administration, especially topical application to skin, is the most preferable mode for the present compositions.

The correct dosage of the pharmaceutical compositions of this invention will vary according to the particular formulation, the mode of application, age, body weight and sex of the patient, diet, time of administration, condition of the patient, drug combinations, reaction sensitivities and severity of the disease. It is understood that the ordinary skilled physician will readily be able to determine and prescribe a correct dosage of this pharmaceutical compositions. An exemplary daily dosage unit for human host comprises an amount of from about 0.001 mg/kg to about 100 mg/kg.

According to the conventional techniques known to those skilled in the art, the pharmaceutical compositions of this invention can be formulated with pharmaceutical acceptable carrier and/or vehicle as described above, finally providing several forms including a unit dosage form. Non-limiting examples of the formulations include, but not limited to, a solution, a suspension or an emulsion, an extract, an elixir, a powder, a granule, a tablet, a capsule, emplastra, a liniment, a lotion and an ointment.

The pharmaceutical composition of this invention may be administered for treating gastric ulcers and neurodegenerative disorders (U.S. Pat. No. 5,200,396; e.g., Parkinson's disease) and wound healing, as recognized by those skilled in the art with regard to EGF.

The following specific examples are intended to be illustrative of the invention and should not be construed as limiting the scope of the invention as defined by appended claims.

### EXAMPLES

### Example I

### Preparation of Novel Gene Encoding EGF

The present gene coding for naturally occurring EGF consisting of 53 amino acids was chemically synthesized (Plant Biotechnology Institute, National Research Centre, Saskatoon, SK, Canada). The nucleotide sequence synthesized, which is indicated in SEQ ID NO:1, is different from that of EGF gene being known.

### Example II

### Preparation of Albumin-EGF Fusion Gene

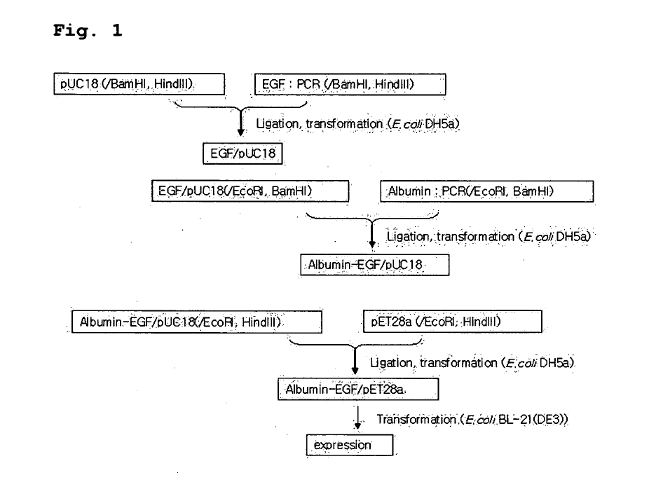

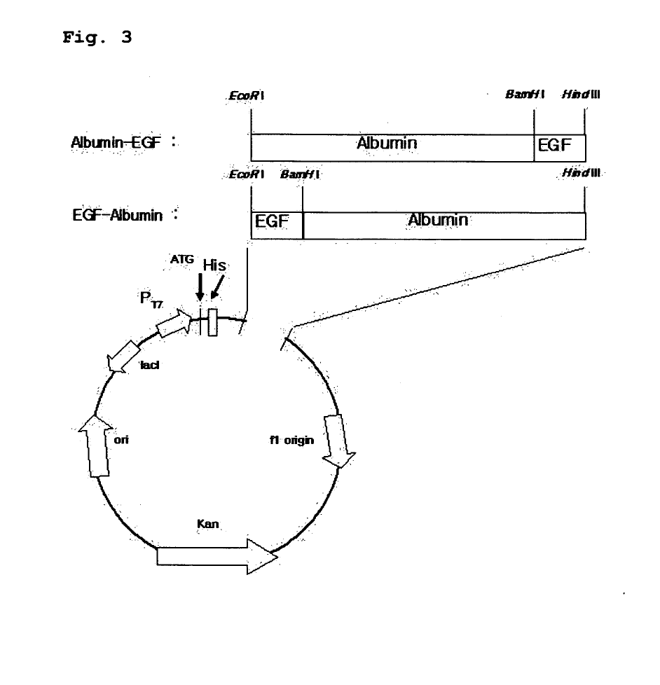

To amplify the EGF gene of Example I, PCR amplification was performed using the EGF gene as template and a pair of primers designed to introduce BamHI and HindIII recognition sites into 5′- and 3′- termini of the gene, respectively. The nucleotide sequences of primers are: reverse primer 5′-CCC AAG CTT TCA GCG CAG TTC CCA CCA CTT-3′; and forward primer 5′-CGG GAT CCA ACA GCG ATT CAG AAT GTC CAC-3′. The PCR product was digested with BamHI and HindIII and extracted. The EGF gene extracted and purified was ligated to pUC18 (Clontech, USA) digested with BamHI and HindIII using T4 DNA ligase (KOSCO CO., KOREA). The resulting vector was transformed into CaCl₂-treated E. coli DH5α (Clontech, USA) and then the transformed cells with ampicillin resistance were selected by culturing in LB medium containing ampicillin (100 mg/ml). The cloned plasmids (EGF/pUC18) were isolated from the transformed cells and then the existence of EGF gene was verified (FIGS. 1 and 3).

PCR amplification was performed using cDNA of human serum albumin as template and a pair of primers designed to introduce EcoRI and BamHI recognition sites into 5′- and 3′- termini of the gene, respectively. The nucleotide sequences of primers are: reverse primer 5′-CGG GAT CCA CCG GTA CGC GTA GAA TCG AGA CC-3′; and forward primer 5′-CGG AAT TCA TGA AGT GGG TAA CCT TTA TTT CC-3′. The PCR product was digested with EcoRI and BamHI and extracted. The human serum albumin gene extracted and purified was ligated to EGF/pUC18 digested with EcoRI and BamHI using T4 DNA ligase. The resulting plasmid was introduced into CaCl₂-treated E. coli DH5α and then the transformed cells with ampicillin resistance were selected by culturing in LB medium containing ampicillin (100 mg/ml). The cloned plasmids (Albumin-EGF/pUC18) were isolated from the transformed cells and then the existence of albumin-EGF fusion gene was verified (FIGS. 1 and 3).

Following the digestion of Albumin-EGF/pUC18 plasmid with EcoRI and HindIII, the resultant was subject to electrophoresis on agarose gel and the albumin-EGF fusion gene was extracted and purified. The albumin-EGF fusion gene purified was ligated to pET28α treated with EcoRI and HindIII using T4 DNA ligase. The resulting plasmid was introduced into CaCl₂-treated E. coli BL21 (DE3) (Clontech, USA) and then the transformed cells with ampicillin resistance were selected by culturing in LB medium containing kanamycin (100 mg/ml). The cloned plasmids (Albumin-EGF/pET28α) were isolated and then the existence of albumin-EGF gene fused in frame was verified by sequencing (FIGS. 1 and 3).

### Example III

### Preparation of EGF-Albumin Fusion Gene

To amplify the EGF gene of Example I, PCR amplification was performed using the EGF gene as template and a pair of primers designed to introduce BamHI and HindIII recognition sites into 5′- and 3′-termini of the gene, respectively. The nucleotide sequences of primers are: reverse primer 5′-CGG GAT CCG CGC AGT TCC CAC CAC TTA AG-3′; and forward primer 5′-CGG AAT TCA TGA ACA GCG ATT CAG AAT GTC CA-3′. The PCR product was digested with BamHI and HindIII and extracted. The EGF gene extracted and purified was ligated to pUC18 digested with BamHI and HindIII using T4 DNA ligase. The resulting vector was transformed into CaCl₂-treated E. coli DH5α and then the transformed cells with ampicillin resistance were selected by culturing in LB medium containing ampicillin (100 mg/ml). The cloned plasmids (EGF/pUC18) were isolated from the transformed cells and then the existence of EGF gene was verified (FIGS. 1 and 3).

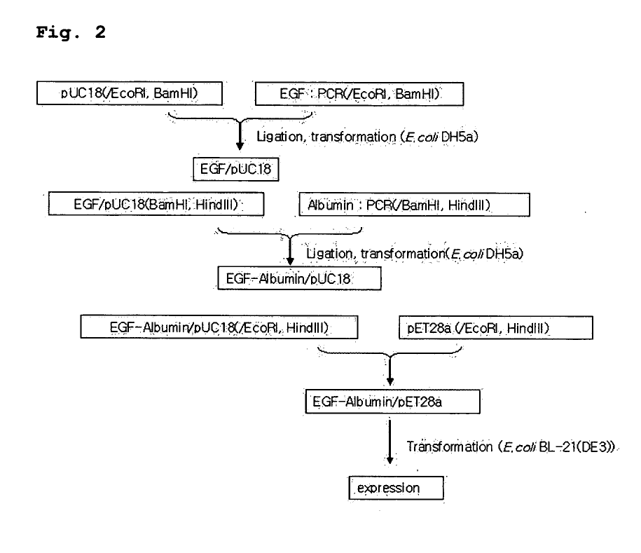

PCR amplification was performed using cDNA of human serum albumin as template and a pair of primers designed to introduce BamHI and HindIII recognition sites into 5′- and 3′-termini of the gene, respectively. The nucleotide sequences of primers are: reverse primer 5′-CCC AAG CTT TCA ACC GGT ACG CGT AGA ATC-3′; and forward primer 5′-CGG GAT CCA AGT GGG TAA CCT TTA TTT CCC-3′. The PCR product was digested with BamHI and HindIII and extracted. The human serum albumin gene extracted and purified was ligated to EGF/pUC18 digested with BamHI and HindIII using T4 DNA ligase. The resulting plasmid was introduced into CaCl₂-treated E. coli DH5α and then the transformed cells with ampicillin resistance were selected by culturing in LB medium containing ampicillin (100 mg/ml) . The cloned plasmids (EGF-Albumin/pUC18) were isolated from the transformed cells and then the existence of EGF-albumin fusion gene was verified (FIGS. 2 and 3).

Following the digestion of EGF-Albumin/pUC18 plasmid with EcoRI and HindIII, the resultant was subject to electrophoresis on agarose gel and the EGF-albumin fusion gene was extracted and purified. The EGF-albumin fusion gene purified was ligated to pET28α treated with EcoRI and HindIII using T4 DNA ligase. The resulting plasmid was introduced into CaCl₂-treated E. coli BL21 (DE3) and then the transformed cells with ampicillin resistance were selected by culturing in LB medium containing kanamycin (100 mg/ml). The cloned plasmids (EGF-Albumin/pET28α) were isolated and then the existence of EGF-albumin gene fused in frame was verified by sequencing (FIGS. 2 and 3).

### Example IV

### Purification of Fusion Protein

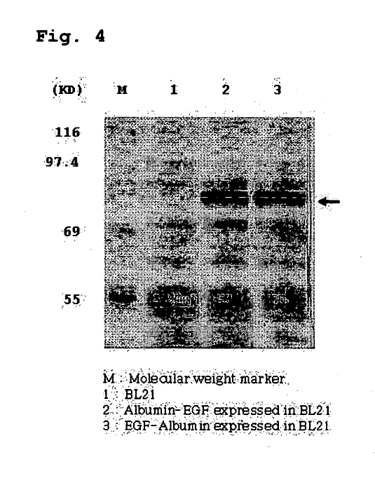

E. coli BL21 (DE3) transformed with Albumin-EGF/pET28α or EGF-Albumin/pET28α was cultured to OD₆₅₀ 0.5 in 5 L fermenter and the expression of the fused gene was then induced by addition of 0.5 mM IPTG (Duchefa, Netherland). Following additional culture for 5-6 hr, the cells were collected by centrifugation. The collected cells were completely suspended in 40 ml of buffer (50 mM Tris, pH 8.0, 1 mM EDTA), disrupted by ultrasonification, centrifuged and the resulting supernatant was then collected. The supernatant was electrophoresed on 8% polyacrylamide gel to verify the expression of the fusion protein (FIG. 4).

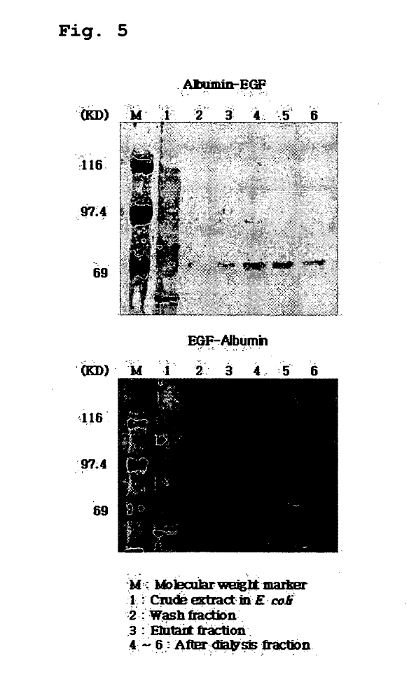

The supernatant was applied to Ni-agarose column (Qiagen, Germany) activated with a binding buffer (20 mM phosphate, 0.5 M NaCl, 10 mM imidazole) and passed at a rate of 1-3 ml/min. Then, using the binding buffer, the column was washed and each of 20, 40, 60, 100, 300 and 500 mM imidazole solutions (pH 7.4) was applied to the column in a stepwise manner, finally eluting the fusion protein, albumin-EGF or EGF-albumin. FIG. 5 shows the results of electrophoresis on 8% polyacrylamide gel of the fraction containing the fusion protein.

### Example V

### Verification of Fusion Protein by Western Blotting

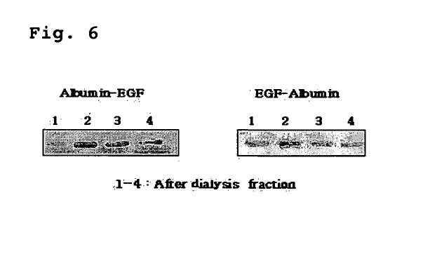

The band of the fusion protein (albumin-EGF or EGF-albumin) on polyacrylamide gel in Example IV was transferred to PVDF membrane and the membrane was incubated for 1 hr with a primary antibody (anti EGF-rabbit, 1:1000 dilution, Santa Cruz, USA). Then, the membrane was incubated for 1 hr with a secondary antibody (rabbit-goat horse radish peroxidase, 1:1000 dilution, KPL, USA) and rinsed. The band was developed using 4-chloro-1-napthol developing solution to verify the existence of the fusion protein (FIG. 6).

### Example VI

### Construction of Expression Vector for Plant Carrying Albumin-EGF Fusion Gene

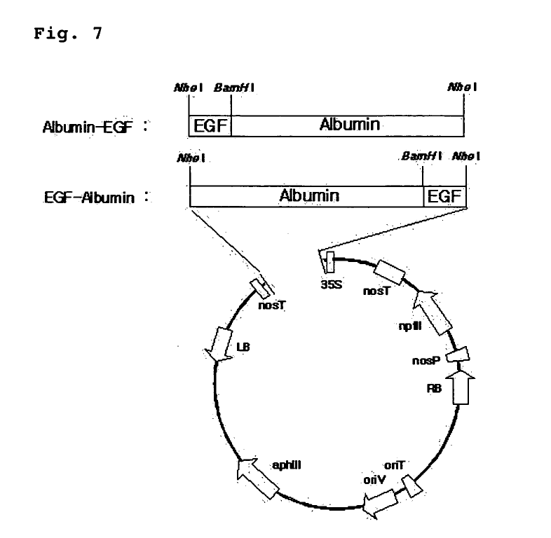

To amplify the fused albumin-EGF gene, a pair of primers was designed and synthesized: forward primer, 5′-CTAGCTAGCGATGAAGTGGGTAACCTTTAT-3′; and reverse primer, 5′-CTAGCTAGCCGCAGTTCCCACCACTTAAGA-3′. The forward primer was designed to have a start codon of albumin gene and NheI restriction site and the reverse primer was designed to have a stop codon of EGF gene and NheI restriction site. 25 μl of PCR mixture was prepared containing 1.25 unit Taq DNA polymerase (Boehringer Mannheim), 2.5 μl of 10x buffer (Boehringer Mannheim), 2 μl of 2.5 mM dNTP, 0.25 μl of 100 pM primers and 50 ng of the fused albumin-EGF gene which was prepared in Example II. The PCR was conducted using Minicycle™ (MJ Research Inc., USA) under the following conditions: pre-denaturation at 95° C. for 2 min followed by 30 cycles of annealing at 55° C. for 1 min, extension at 720° C. for 1 min and denaturation at 92° C. for 1 min; followed by final extension at 72° C. for 10 min. Amplified products were analyzed by electrophoresis on 0.8% TAE agarose gel at the constant temperature of 4° C. The fused albumin-EGF gene was eluted and obtained from the corresponding band. The fused albumin-EGF gene purified was digested with NheI and inserted into binary vector pRD400 (Raju et al., Gene 211: 383-384(1992)) digested with XbaI, finally constructing the expression vector of albumin-EGF gene for plant (FIG. 7).

### Example VII

### Construction of Expression Vector for Plant Carrying EGF-Albumin Fusion Gene

To amplify the fused albumin-EGF gene, a pair of primers was designed and synthesized: forward primer, 5′-CTAGCTAGCGATGAACAGCGATTCAGAATG-3′; and reverse primer, 5′-CTAGCTAGCCCGGTACGCGTAGAATCGAGA-3′. The forward primer was designed to have a start codon of EGF gene and NheI restriction site and the reverse primer was designed to have a stop codon of albumin gene and NheI restriction site. The PCR amplification was conducted according to the same manner as Example VI. The EGF-albumin gene obtained was digested with NheI and inserted into binary vector pRD400 digested with XbaI, thus constructing the expression vector of EGF-albumin gene for plant (FIG. 7).

### Example VIII

### Transformation of Plant

### Example VIII-1

Preparation of Transformed Agrobacterium tumefaciens

The expression vector, pRD400::(albumin-EGF) of Example VI or pRD400::(EGF-albumin) of Example VII was introduced into Agrobacterium tumefaciens (Agrobacterium tumefaciens GV3101(Mp90); Plant-cell-rep., 15(11)799-803(1996)) by means of conjugation. To select Agrobacterium tumefaciens harboring the expression vector, the incubated mixture for conjugation was spread on LB solid medium containing 50 mg/L of kanamycin and 30 mg/L of gentamicin and incubated for 2 days at 28° C. The selected Agrobacterium tumefaciens was inoculated into super broth (BHI medium, pH 5.6) and incubated for 2 days at 28° C.

### Example VIII-2

Transformation of Cucumis melo

The seeds of Cucumis melo sterilized with 1% NaOCl solution were seeded for obtaining cotyledons. The cotyledons were collected in a manner that their growth points were completely removed. Agrobacterium tumefaciens transformed with pRD400::(albumin-EGF) or pRD400::(EGF-albumin) was incubated for 18 hr at 28° C. in super broth containing 100 μM acetosyringone (37 g/l brain heart infusion broth(Difco) and 0.2% sucrose, pH 5.6), and then the resulting medium was diluted 20-fold with inoculation medium. The above inoculation medium (pH 5.6) contains MSB5 (Murashige & Skoog medium including Gamborg B5 vitamins), 3.0% sucrose, 0.5 g/L of MES [2-(N-Morpholino)ethanesulfonic acid Monohydrate], 6.0 mg/L of kinetin, 1.5 mg/L of IAA (indole-3-acetic acid), 1.0 mg/L of CuSO₄·5H₂O, 100 μM acetosyringone and 5% DMSO.

Thereafter, the cotyledon was immersed in 40 ml of the inoculation medium and incubated for 20 min to inoculate with Agrobacterium tumefaciens . Then, the cotyledon was transferred to a coculturing medium with its outface being upward. The coculturing medium contains MSB5, 3.0% sucrose, 0.5 g/L of MES, 6.0 mg/L of kinetin, 1.5 mg/L of IAA, 1.0 mg/L of CuSO₄·5H₂O, 0.6% agar, 100 μM acetosyringone and 5% DMSO. The cotyledon was then cocultured under dark culture condition (26±1° C., 24 hrs night) for 3 days. After coculturing, in order to form shoots by regeneration from cotyledon and select transformed shoots, the cotyledon was placed on a selection medium and cultured at 25±1° C. and 4,000 lux under 16 hr light condition to induce generation of shoots. The selection medium (pH 5.6) contains MSB5, 3.0% sucrose, 0.5 g/L of MES, 6.0 mg/L of kinetin, 1.5 mg/L of IAA, 1.0 mg/L of CuSO₄·5H₂O, 0.6% agar, 100 mg/L of kanamycin and 500 mg/L of carbenicillin. Then, the regenerated shoots were transferred to a fresh selection medium followed by light culture for 2 weeks.

Thereafter, the elongated shoots were transferred to a rooting medium and cultured for 2 weeks. The shoots with roots, which were considered to be transformed, were selected. The rooting medium (pH 5.6) contains MSB5, 3.0% sucrose, 0.5 g/L of MES, 0.1 mg/L of NAA (α-naphtalene acetic acid), 1.0 mg/L of CuSO₄·5H₂O, 0.6% agar, 100 mg/L of kanamycin and 500 mg/L of carbenicillin.

### Example VIII-3

Transformation of Curcumis sativa

The seeds of Curcumis sativa sterilized with 1% NaOCl solution were seeded for obtaining cotyledons. The cotyledons were collected in a manner that their growth points were completely removed. Agrobacterium tumefaciens transformed with pRD400::(albumin-EGF) or pRD400::(EGF-albumin) was incubated in the same manner as Example VIII-2. The cotyledon was immersed for 10 min in the inoculation medium containing the same ingredients as Example VIII-2.

Thereafter, the cotyledon was cultured in a coculturing medium containing MSB5, 2 mg/L of BAP and 0.01 mg/L of NAA under light culture condition at 26° C. for 2 days and then was cocultured with Agrobacterium tumefaciens at 4° C. for 4 days. After coculturing, the cotyledon was placed on a selection medium and cultured at 26±1° C. and 8,000 lux under 16 hr light/8 hr dark condition. The selection medium (pH 5.6) contains MSB5, 3.0% sucrose, 0.5 g/L of MES, 0.4% phytagel, 2 mg/L of BAP, 0.01 mg/L of NAA, 500 mg/L of carbenicillin and 100 mg/L of kanamycin. Then, the regenerated shoots were transferred to a rooting medium (containing 0.01 mg/L of NAA, 100 mg/L of kanamycin and 0.4% agar) and cultured at 26±1° C. and 8,000 lux under 16 hr light/8 hr dark condition. The shoots with roots, which were considered to be transformed, were selected.

### Example VIII-4

Transformation of Citrullus vulgaris

The seeds of Citrullus vulgaris sterilized with 1% NaOCl solution were seeded for obtaining cotyledons. The cotyledons were collected in a manner that their growth points were completely removed. Agrobacterium tumefaciens transformed with pRD400::(albumin-EGF) or pRD400::(EGF-albumin) was incubated in the same manner as Example VIII-2. The cotyledon was immersed for 10 min in the inoculation medium containing the same ingredients as Example VIII-2.

Thereafter, the cotyledon was placed on a coculturing medium (pH 5.6) containing 4.04 g/L of MSB5, 2 mg/L of BAP, 0.5 g/L of MES and 0.6% agar and cultured under 16-hour light culture condition at 25±1° C. and 4,000 lux for 2 days. Cultured cotyledon was placed on the regeneration medium (pH 5.6) containing MSB5, 2 mg/L of BAP, 3.0% sucrose, 0.5 g/L of MES, 0.4% phytagel, 500 mg/L of carbenicillin and 200 mg/L of kanamycin and pre-cultured at 25° C.±1° C. for 7 days to induce generation of shoots. Then, the shoots induced were cultured in the selection medium containing 200 mg/L of kanamycin for 4 weeks to select the shoots with roots, which were considered to be transformed.

### Example VIII-5

Transformation of Brassica campestris

The seeds of Brassica campestris sterilized were seeded for obtaining petiole. The petioles were collected in a manner that their growth points were completely removed. Agrobacterium tumefaciens transformed with pRD400::(albumin-EGF) or pRD400::(EGF-albumin) was incubated in the same manner as Example VIII-2. The petiole was immersed for 10 min in the inoculation medium containing the same ingredients as Example VIII-2.

Thereafter, the petiole was cultured in a coculturing medium (pH 5.8) containing MSB5, 3% sucrose, 1 mg/L of 2,4-D and 6.5 g/L of agar power at 25° C. for 2 days and subsequently at 4° C. for 4 days. To select the transformed Brassica campestris, the petiole was transferred to a selection medium and cultured at 25° C. for 2 weeks under 16-hr light/8-hr dark condition. The selection medium (pH 5.8) contains MSB5, 3% sucrose, 5 g/L of MES, 2 mg/L of BAP, 0.01 mg/L of NAA, 20 mg/L of kanamycin, 500 mg/L of Psedopen and 6.5 g/L of agar power. The root for shoot was induced in a rooting meduium (pH 5.8) containing MSB5, 3.0% sucrose, 5 g/L of MES, 0.1 mg/L of NAA, 20 mg/L of kanamycin 500 mg/L of Pseudopen and 6.5 g/L of agar.

### Example VIII-6

Transformation of Nicotiana tabacum

The seeds of Nicotiana tabacum were seeded and cultivated in sterilized condition for obtaining young leaves. Agrobacterium tumefaciens transformed with pRD400::(albumin-EGF) or pRD400::(EGF-albumin) was incubated in the same manner as Example VIII-2 and then mixed with the inoculation medium of Example VIII-2. The fragments of young leaf with a size of 0.5-1 cm² were immersed for 10-15 min in the inoculation medium and then transferred to a coculturing medium (pH 5.8) containing MSB5, 3.0% sucrose, 0.5 g/L of MES, 1.0 mg/L of BAP, 0.1 mg/L of NAA and 0.6% agar.

The fragment was cocultured under dark culture condition (26±1° C., 24 hrs night) for 2 days. After coculturing, in order to form shoots by regeneration and select transformed shoots, the fragment was placed on a selection medium and cultured at 26±1° C. and 4,000 lux for 2 weeks under 16-hr light condition. The selection medium (pH 5.6) contains MSB5, 3.0% sucrose, 0.5 g/L of MES, 1.0 mg/L of BAP, 0.1 mg/L of NAA, 0.6% agar, 100 mg/L of kanamycin and 500 mg/L of carbenicillin. Thereafter, the elongated shoots were transferred to a rooting medium and cultured for 2 weeks. The shoots with roots, which were considered to be transformed, were selected. The rooting medium (pH 5.6) contains MSB5, 3.0% sucrose, 0.5 g/L of MES, 0.01 mg/L of NAA, 0.6% agar, 100 mg/L of kanamycin and 500 mg/L of carbenicillin.

### Example IX

### Verification on Transformation of Plant

The transformants in Example VIII were verified as described below:

Using ten mg of the shoots rooted that were considered to be transformed, a genomic DNA for PCR analysis was obtained according to the method described by Edwards K., et al. (Nucleic Acids Research, 19: 1349(1991)) and then PCR analysis was performed.

The primer set for PCR analysis of plant transformed with albumin-EGF fusion gene is: forward primer, 5′-CTAGCTAGCGATGAAGTGGGTAACCTTTAT-3′; and reverse primer, 5′-CTAGCTAGCCGCAGTTCCCACCACTTAAGA-3′.

The primer set for PCR analysis of plant transformed with EGF-albumin fusion gene is: forward primer, 5′-CTAGCTAGCGATGAACAGCGATTCAGAATG-3′; and reverse primer, 5′-CTAGCTAGCCCGGTACGCGTAGAATCGAGA-3′.

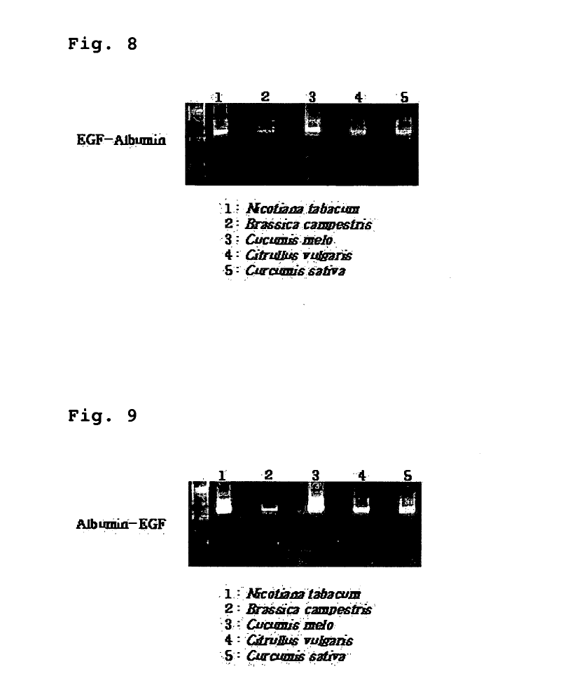

The PCR amplification was conducted using Taq polymerase according to the following thermal conditions: pre-denaturation at 96° C. for 2 min followed by 35 cycles of annealing at 55° C. for 1 min, extension at 72° C. for 2 min and denaturation at 94° C. for 1 min; followed by final extension at 72° C. for 10 min. Amplified products were analyzed by electrophoresis on 1.0% agarose gel (FIGS. 8 and 9).

In FIG. 8, lane M shows 1 kb ladder, lanes 1, 2, 3, 4 and 5 represent PCR products of transformed Nicotiana tabacum, Brassica campestris, Cucumis melo, Citrullus vulgaris and Curcumis sativa, respectively. As shown in FIG. 8, the bands corresponding to albumin-EGF fusion gene (2088 bp) are observed in each lane.

In FIG. 9, lane M shows 1 kb ladder, lanes 1, 2, 3, 4 and 5 represent PCR products of transformed Nicotiana tabacum, Brassica campestris, Cucumis melo, Citrullus vulgaris and Curcumis sativa, respectively. As shown in FIG. 9, the bands corresponding to EGF-albumin fusion gene (2088 bp) are observed in each lane.

Therefore, it is recognized that the plants in Example VIII are transformed with albumin-EGF or EGF-albumin fusion gene and harbor stably the foreign fusion gene.

### Example X

### Verification of Fusion Protein in Plant

Transformants

2.5 ml of extraction buffer (containing 100 mM Tris-Cl, pH 7.5, 500 mM EDTA, pH 8.0, 1 mg/ml leupeptin, 5 mg/ml BSA, 1 mg/ml DTT and 30 mg/ml PMSF) were added to 1 g of the leaves of transformants prepared in Example VIII and then the leaves were ground finely in a mortar. The extract was centrifuged at 12,000 rpm and 4° C. for 30 min, the supernatant was transferred to a new tube and stored on ice.

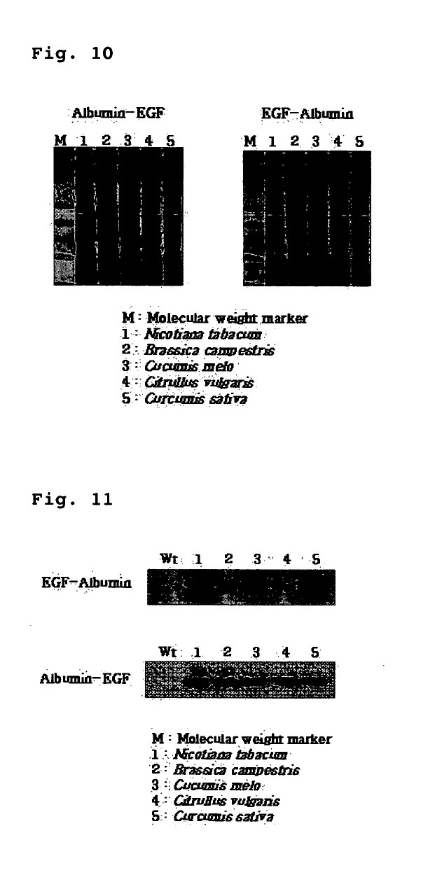

Protein quantification of the extract was performed using protein assay kit (Bio-Rad) in accordance with Bradford method. The extract samples with the same amount were electrophoresed on 8% polyacrylamide gel (FIG. 10). In FIG. 10, lane M shows protein marker, lanes 1, 2, 3, 4 and 5 correspond to the transformed Nicotiana tabacum, Brassica campestris, Cucumis melo, Citrullus vulgaris and Curcumis sativa, respectively. As shown in FIG. 10, the bands corresponding to albumin-EGF fusion protein (70 kDa) were observed in each lane.

The band corresponding to albumin-EGF fusion protein was transferred to PVDF membrane and then the primary antibody (anti EGF-rabbit, 1:1000 dilution, Santa Cruz, USA) was added to PVDF membrane and incubated for 1 hr. After incubation, the membrane was washed and incubated with the secondary antibody (rabbit-goat horseradish peroxidase, 1:1000 dilution) for 1 hr and washed. Then, the color development was allowed with 4-chloro-1-naphtol. As shown in FIG. 11, the bands showing the expected size, i.e., 70 kDa were observed, so that the existence of albumin-EGF fusion protein in transformants was verified.

### Example XI

### Analysis of Expression Level of Fusion Protein in Plant Transformants

To analyze the expression level of fusion protein in plant transformants, the following reagents were prepared: washing buffer: PBST (0.05% Tween 20 and PBS, pH 7.4); diluent buffer: TBST (0.1% BSA, 0.05% Tween 20 and TBS); TBS (20 mM Trisma base, 150 mM NaCl); blocking buffer (1% BSA, 5% sucrose, 0.05% NaN3 in PBS); substrate solution (ABTS peroxidase substrate, KPL corp., USA); stop solution (1% Sodium Dodecyl Sulfate (SDS)); primary antibody (peroxidase labelled-Anti Human IgG, Santa Cruz, USA); and secondary antibody (peroxidase labelled-Anti mouse IgG, Santa Cruz, USA).

The proteins extracted and purified from the plant transformants serially diluted to 78 ng, 36 ng, 18 ng, 9 ng, 4 ng, 2 ng, 1 ng, 576 pg, 288 pg, 144 pg, 72 pg and 36 pg were placed to each well of plate and then kept 4° C. for 8 hr. The plate was washed three times with the washing buffer. Then, the primary antibodies serially diluted to 1/100, 1/200. 1/400, 1/800, 1/1600, 1/3200, 1/6400, 1/12800 were inoculated to each well of the plate and then kept 4° C. for 2 hr, followed by washing with the washing buffer.

Thereafter, 300 μl of blocking buffer were added to each well and then allowed to stand for 2 hr at 4° C. and washed with the washing buffer. The secondary antibodies (1/1000 dilution) were added to each well and incubated for 1 hr at 4° C., followed by washing with the washing buffer. The substrate was added to each well and incubated for 30 min, after which the reaction was stopped with 50 μl of the stop buffer. Finally, the absorbance at 405 nm was measured with ELISA reader (TECAN sunrise)

TABLE I

| | Expression level (pg/g) | | | |
|---|---|---|---|---|
| | Wild | Transformant | | |
| Plant | type | EGF | Albumin-EGF | EGF-albumin |
| Cucumis melo | 0 | 31.70 | 36.43 | 41.10 |
| Curcumis sativa | 0 | 25.09 | 27.24 | 33.10 |
| Citrullus vulgaris | 0 | 32.00 | 14.40 | 36.10 |
| Brassica campestris | 0 | 39.01 | 45.56 | 49.70 |
| Nicotiana tabacum | 0 | 42.06 | 17.95 | 40.90 |

As shown in Table I, the transformants with the nucleotide sequence coding for the fusion protein comprising EGF and human serum albumin linked to the C-terminal of EGF exhibit the highest expression level.

Example XII: Analysis of Stability of Fusion Protein in Plant Transformants

Firstly, the purification of the fusion protein in plant trasnformants was carried out as follows: The tissues from the plant transformants were grinded in the homogenization buffer (250 mM sucrose, 1 M Hepes, 1 mM DTT and 1 mM MgCl₂) in the presence of liquid nitrogen and then centrifuged for 10 min at 7000×g and 4° C., followed by collecting supernatant. The centrifugation was performed once more under the same conditions. The extract was mixed with 1 ml (bed volume) of preequilibrated Qiagen resin and then gentle stirred for 1 hr in the cold. The resultant was poured into Ni-NTA agarose column (Qiagen, Germany) . Thereafter, the column was washed with column volumes of the elution buffer (0.3 M NaCl, 20 mM BME, 250 mM imidazole, and 50 mM Na-phosphate buffer pH 8.0). The eluant was dialyzed in 500 ml for 2 to 4 hr in dialysis buffer (40 mM Hepes, pH 8.0, 200 mM NaCl and 1 mM DTT) with two changes. Following dialysis, the concentration of the fractions was checked with with Protein assay kit (BioRad, USA). The fraction containing the fusion protein was subject to SDS-PAGE and stained with coomassie blue to check its purity. Furthermore, Western Blotting was performed.

The stability of the fusion protein obtained from the plant transformants was examined according to the following procedures: The capture antibody (mouse monoclonal rhEGF IgG) was diluted in PBS at a rate of 1/1000 and 100 μl of the diluent then placed to each well of the plate for ELISA, followed by keeping for 8 hr at a room temperature. The plate was washed three times with washing buffer and then was added with 300 μl of blocking buffer, followed by allowing to stand for 2 hr. 100 μl of each of the present fusion protein and the standard protein (human EGF, KOMA, Korea) were added to the plate and mixed thoroughly, followed by storing at a room temperature or 4° C. for more than 8 hr. Thereafter, washing of the plate was performed three times, and 100 μl of the detection antibody (biotinylated EGF affinity purified Goat IgG, 1/1000 dilution) were placed to each well of the plate, followed by allowing to stand for 2 hr with stirring. After washing, 100 μl of streptavidin-HRP (R&D systems) diluted to 1:50 in PBS were added to each well and incubated for 1 hr, followed by washing. Then, the substrate buffer diluted to 1:4 in PBS was added to each well and incubated to proceed the reaction. 50 μl of the stop solution was added to each well to terminate the reaction and then the absorbance at 540-570 nm was measured.

The results of stability analysis are summarized in Table II.

TABLE II

| pH | Temp (° C.) | EGF | Albumin-EGF | EGF-albumin |
|---|---|---|---|---|
| 7.0 | 4 | 100 | 100 | 99.8 |
| | 25 | 43.0 | 75.0 | 91.0 |
| | 45 | 0 | 13.7 | 77.6 |
| 4.7 | 4 | 100 | 100 | 100 |
| | 25 | 56.9 | 91.2 | 96.7 |
| | 45 | 0 | 0 | 94.8 |

As indicated in Table II, EGF in the fusion protein of this invention is revealed to show higher stability than non-fused EGF. In particular, EGF linked to the N-terminal of albumin exhibits excellent stability under any storage condition.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A method for preparing a fusion polypeptide comprising epidermal growth factor (EGF) and human serum albumin in a plant, which comprises the steps of:
(a) transforming plant cells with a polynucleotide sequence comprising:
(i) a nucleotide sequence encoding said fusion polypeptide comprising EGF and human serum albumin linked to the C-terminal or N-terminal of said EGF; and in which the stability of said EGF is enhanced by virtue of said human serum albumin;
(ii) a promoter that functions in plant cells to cause the production of an RNA molecule operably linked to the nucleotide sequence of (i); and
(iii) a 3′-non-translated region that functions in plant cells to cause the polyadenylation of the 3′-end of said RNA molecule;
(b) selecting transformed plant cells;
(c) regenerating a plant from said transformed cells; and
(d) recovering from said regenerated plant said fusion polypeptide.

2. The method according to claim 1, wherein said plant is Nicotiana tabacum, Cucumis melo, Curcumis sativa, Citrullus vulgaris or Brassica campestris.

3. The method according to claim 1, wherein a nucleotide sequence of said EGF comprises nucleotide 1-159 as set forth in SEQ ID NO:1.

4. The method according to claim 1, wherein said human serum albumin is linked to the C-terminal of said EGF.

5. A method for preparing a fusion polypeptide comprising EGF and human serum albumin in a plant, which comprises the steps of:
(a) inoculating an explant material from said plant with Agrobacterium tumefaciens harboring a vector, in which said vector is capable of inserting into a genome of a cell from said plant and contains the following nucleotide sequences:
(i) a nucleotide sequence encoding said fusion polypeptide comprising EGF and human serum albumin linked to the C-terminal or N-terminal of said EGF; and in which the stability of said EGF is enhanced by virtue of said human serum albumin;
(ii) a promoter that functions in plant cells to cause the production of an RNA molecule operably linked to the nucleotide sequence of (i); and
(iii) a 3′-non-translated region that functions in plant cells to cause the polyadenylation of the 3′-end of said RNA molecule;
(b) regenerating the inoculated explant material on a regeneration medium to obtain regenerated shoots;
(c) culturing said regenerated shoots on a rooting medium to obtain a transformed plant, in which said transformed plant is capable of expressing said nucleotide sequence of (i); and
(d) recovering from said transformed plant said fusion polypeptide.

6. The method according to claim 5, wherein said plant is Nicotiana tabacum, Cucumis melo, Curcumis sativa, Citrullus vulgaris or Brassica campestris.

7. The method according to claim 5, wherein a nucleotide sequence of said EGF comprises nucleotide 1-159 as set forth in SEQ ID NO:1.

8. The method according to claim 5, wherein said human serum albumin is linked to the C-terminal of said EGF.
